(19) United States
(12) Patent Application Publication
(10) Pub. No.: US 2026/0085159 A1
(43) Pub. Date: Mar. 26, 2026

(54) RADIOPAQUE POLYPROPYLENE

(71) Applicant: Boston Scientific Scimed, Inc., Maple Grove, MN (US)

(72) Inventors: Heather Cirka, Hudson, MA (US); Yen-Hao Hsu, Shrewsbury, MA (US)

(21) Appl. No.: 19/331,793

(22) Filed: Sep. 17, 2025

Related U.S. Application Data
(60) Provisional application No. 63/698,189, filed on Sep. 24, 2024.

(51) Int. Cl. C08J 3/20; A61L 31/04; A61L 31/18; B29C 48/05; B29K 25/00; B29L 31/00; C08K 3/30

(52) U.S. Cl. CPC C08J 3/201; A61L 31/048; A61L 31/18; B29C 48/05; C08K 3/30; B29K 2025/08; B29K 2995/0025; B29L 2031/753; C08J 2323/12; C08J 2425/18; C08K 2003/3045

(57) ABSTRACT

According to an aspect, a method includes forming a material including polypropylene and a radiopaque component and using the material to form a medical device. In some implementations, the medical device includes a mesh. In some implementations, the medical device includes a knitted mesh. In some implementations, the using the material includes extruding the material to form a filament. In some implementations, the forming the material includes forming a plurality of solid pellets of the material. In some implementations, the forming the material includes forming a plurality of solid pellets of the material, and the using the material includes placing at least some of the plurality of solid pellets into a mold to form the medical device.

### CROSS-REFERENCE TO RELATED APPLICATION

This application claims priority to U.S. Provisional Patent Application No. 63/698,189, filed on Sep. 24, 2024, entitled “RADIOPAQUE POLYPROPYLENE”, the disclosure of which is incorporated by reference herein in its entirety.

### TECHNICAL FIELD

The present invention generally relates to medical devices and materials that are used to make or form medical devices that are radiopaque.

### BACKGROUND

Medical devices, such as bodily implants, are used to treat the body of a patient in many circumstances. For example, bodily implants may be used to provide support or structure within a body of a patient. In some examples, a woven or mesh material may be used to treat incontinence, such as urinary incontinence. Other uses of surgically implantable devices such as meshes include meshes for hernia repair (e.g., meshes for inguinal hernia, hiatus hernia, etc.), meshes for thoracic wall defects, breast support meshes and various other soft-tissue surgical mesh support devices, including meshes for cosmetic and reconstructive surgery, among others. In other examples, bodily implants other than knitted or woven meshes may be used to treat the body of the patient in different ways.

In some cases, the medical devices are observed during an implantation procedure or after an implantation procedure. Accordingly, there is a need for bodily implants and materials that may form bodily implants that include radiopaque materials or are otherwise radiopaque.

### SUMMARY

According to an aspect, a method includes forming a material including polypropylene and a radiopaque component and using the material to form a medical device. In some implementations, the medical device includes a mesh. In some implementations, the medical device includes a knitted mesh.

In some implementations, the using the material includes extruding the material to form a filament. In some implementations, the forming the material includes forming a plurality of solid pellets of the material. In some implementations, the forming the material includes forming a plurality of solid pellets of the material, the using the material includes placing at least some of the plurality of solid pellets into a mold to form the medical device.

In some implementations, the medical device includes a molded medical device.

In some implementations, the radiopaque component includes iodine. In some implementations, the forming the material includes compounding polypropylene with the radiopaque component. In some implementations, the forming the material includes compounding the polypropylene with barium sulfate. In some implementations, the forming the material includes compounding the polypropylene with barium sulfate to form a plurality of solid pellets.

In some implementations, the forming the material includes forming a polypropylene-iodinated polystyrene co-polymer.

In some implementations, the material includes a co-polymer having the following formula

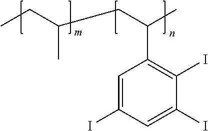

where m and n are integers.

In some implementations, the forming the material includes copolymerizing propylene with iodinated styrene. In some implementations, the forming the material includes copolymerizing propylene with iodinated styrene utilizing a catalyst.

According to another aspect, a method includes forming a co-polymer having the following formula

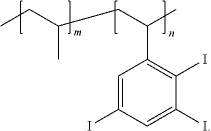

In some implementations, the forming the co-polymer includes copolymerizing propylene with iodinated styrene. In some implementations, the forming the co-polymer includes copolymerizing propylene with iodinated styrene utilizing a catalyst. In some implementations, the method further includes using the co-polymer to form a medical device.

According to another aspect, a material includes a co-polymer having the following formula

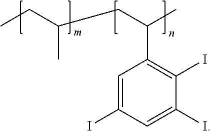

### BRIEF DESCRIPTION OF THE DRAWINGS

FIG. 1 is a top view of a medical device according to an implementation.

FIG. 2 is a top view of another medical device according to an implementation.

FIG. 3 is a flow chart of a method according to an implementation.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

The present invention relates to medical devices such as bodily implants that include or are formed with radiopaque materials. The present invention also relates to materials that include radiopaque materials that may be used to make or form medical devices. In some cases, the medical devices of the present invention may be implanted within a body of a patient and may be viewed or visualized, for example, using an x-ray device or computed tomography system. In some cases, the medical device may be viewed during an implantation procedure (for example, to confirm correct placement) or after an implantation procedure has been completed (for example, to confirm that the medical device has not migrated within the body of the patient).

Various embodiments, aspects, or implementations of the medial device are described herein. They should be considered as exemplary and should not be used to limit the scope of the invention.

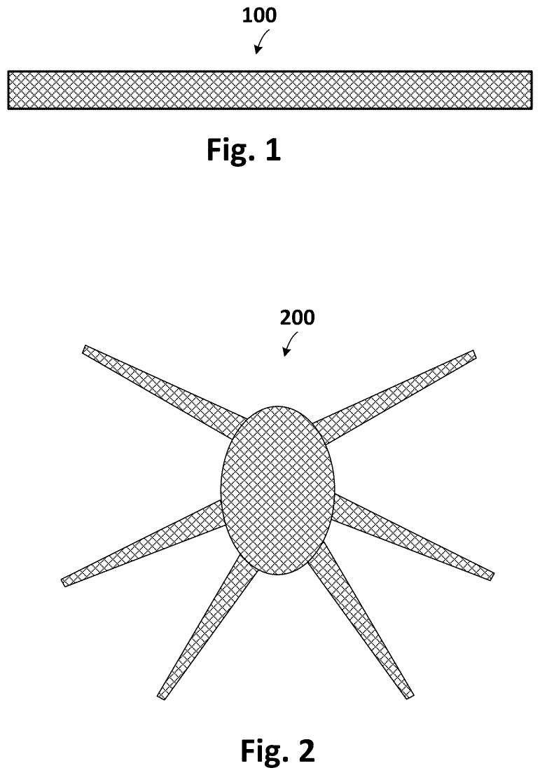

FIG. 1 is a top view of a medical device 100. The medical device 100 is a mesh such as a woven or knitted mesh. For example, the mesh may be formed by weaving or knitting a plurality of strands of material. In some implementations, the medical device 100 may be placed within the body of a patient and be configured to provide support to a portion of the body of the patient. For example, in some examples, the medical device 100 is a sling, such as a suburethral sling. While the medical device 100 is illustrated as having a rectangular shape. In other implementations, the medical device 100 has a different shape. Additionally, in other implementations, the medical device 100 functions within the body of the patient other than providing support to a portion of the body of the patient.

The medical device 100 is formed of a material that includes radiopaque material that is visible using an x-ray device or computed tomography system. For example, in some implementations, the medical device 100 may be placed within a body of a patient and may be viewed by a physician using an x-ray device or computed tomography system or device. In some implementations, the medical device 100 is formed of a polymer material and a radiopaque material.

In some implementations, the medical device 100 is made or formed by extruding strands or filaments of material. The strands or filaments of materials may then be woven or knitted into the mesh that forms the medical device 100. In some implementations, the material that is used to form the medical device 100 is the form of a pellet or a plurality of pellets. The pellets of material may be melted to then be extruded to form the strands or filaments. In some implementations, the material that is used to form the medical device 100 is in the form of a powder or small granules of material.

FIG. 2 is another medical device 200 according to another implementation. The medical device 200 is a molded implant. For example, the molded implant may be formed by placing material within a mold. In some implementations, the medical device 200 may be placed within the body of a patient and be configured to provide support to a portion of the body of the patient. While the medical device 200 is illustrated as having a specific shape, in other implementations, the medical device 200 has a different shape. Additionally, in other implementations, the medical device 200 functions within the body of the patient other than providing support to a portion of the body of the patient.

The medical device 200 is formed of a material that includes radiopaque material that is visible using an x-ray or computed tomography device. For example, in some implementations, the medical device 200 may be placed within a body of a patient and may be viewed by a physician using an x-ray or computed tomography device. In some implementations, the medical device 200 is formed of a polymer material and a radiopaque material.

In some implementations, the medial device 200 is made or formed by placing material within a mold and otherwise exposed to a molding process. In some implementations, the material that is used to form the medical device 200 is the form of a pellet or a plurality of pellets. The pellets of material may be melted to then be used in the mold or the molding process. In some implementations, the material that is used to form the medical device 200 is in the form of a powder or small granules of material.

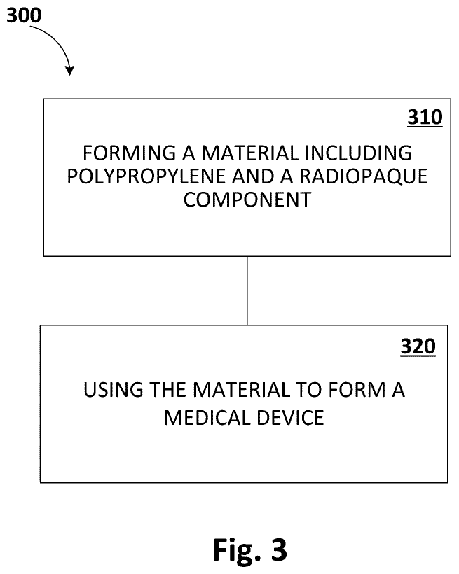

FIG. 3 shows a method 300 according to an implementation. The method includes forming a material that includes polypropylene and a radiopaque component (310). Then, the material is used to form a medical device, such as medical device 100 or medical device 200 (320). In other implementations, the method includes forming a material that includes other polyolefins, such as polyethylene, polyisobutylene, or polystyrene, and a radiopaque component.

In some implementations, material used to make or form a medical device, such as medical device 100 and medical device 200, may be made using a compounding process. For example, different components of the material used to make or form the medical device may be combined using a compounding process. In some cases, the different components may be combined while the components are in a molten state and then may be mixed and cooled. In some implementations, the combined material may then be formed into pellets (which may then be used to make or form medical devise, such as medical device 100 and medical device 200).

In some implementations, the material used to form the medical devices includes a first component and a second component. In some implementations, the first component is a polymer such as polypropylene. In some implementations, the second component is a radiopaque material such as barium sulfate. In other implementations, the second component is a different radiopaque material.

In some implementations, polypropylene and barium sulfate are compounded to form a combination of the polypropylene and barium sulfate. The combined product can be placed in pellet or powder form and can be used to make devices such as medical device 100 and medical device 200.

In some implementations, a chemical bonding process may be used to form the material used to make the medical devices, such as medical device 100 and medical device 200. For example, in some implementations, a co-polymer may be formed and used as the material. In some implementations, the co-polymer has a first component and a second component and a second component that is radiopaque. In some implementations, the co-polymer is polypropylene-iodinated polystyrene. In other implementations, the co-polymer is a different co-polymer. In some implementations, the co-polymer may be placed in pellet or powder form and can be used to make the medical devices.

In some implementations, the co-polymer is polypropylene-iodinated polystyrene and has the below formula,

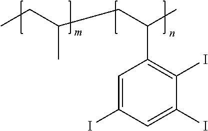

where m and n are integers. In some implementations, the ratio of the integers (m to n) is between 100 (m) to 1 (n) and 5 (m) to 1 (n). In other implementations, the ratio of the integers is different.

In some implementations, polypropylene-iodinated polystyrene co-polymer is formed by combining propylene with iodinated styrene as shown below.

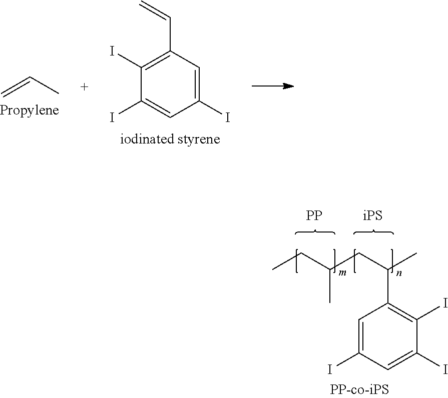

A variety of methods or processes may be used to form polypropylene-iodinated polystyrene co-polymer.

In one implementation, polypropylene-iodinated polystyrene co-polymer is formed using the following process or method. First, polypropylene is made from the polymerization of propylene. For example, in one implementation, propylene gas is condensed into liquid phase at 300-400 K at 30-40 atm in the presence of a Ziegler-Natta catalyst. In some implementations, a ferro-based metal catalyst is used. In some implementations, the polymerization takes place with solvent-free environment. Then, 1-ethenyl-2, 3, 5-triiodo benzene which has been dissolved in an organic solvent (such as dichloromethane (DCM,) methanol, or acetone) is added to further polymerize with the polypropylene segment to form polypropylene-iodinated polystyrene co-polymer. In some implementations, the ratio of repeating units of polypropylene to iodinated polystyrene can be varied. For example, in one implementation, the ratio may be varied depending on the desired radiopacity.

In another implementation, polypropylene-iodinated polystyrene co-polymer is formed using the following process or method. First, polypropylene is made from the polymerization of propylene. For example, in one implementation, propylene gas is condensed into liquid phase at 300-400 K at 30-40 atm in the presence of a Ziegler-Natta catalyst. In some implementations, a ferro-based metal catalyst is used. In some implementations, the polymerization takes place with solvent-free environment. Separately, 1-ethenyl-2, 3, 5-triiodo benzene which has been dissolved in an organic solvent (such as dichloromethane (DCM,) methanol, or acetone) is mixed with a Ziegler-Natta catalyst to form iodinated polystyrene oligomer. The iodinated polystyrene oligomer is then mixed or added to the polypropylene segment to form polypropylene-iodinated polystyrene co-polymer. In some implementations, the ratio of repeating units of polypropylene to iodinated polystyrene can be varied. For example, in one implementation, the ratio may be varied depending on the desired radiopacity.

In another implementation, polypropylene-iodinated polystyrene co-polymer is formed using the following process or method. First, 1-ethenyl-2, 3, 5-triiodo benzene is dissolved in acetone (or another organic solvent) with a Ziegler-Natta catalyst to form an iodinated polystyrene segment. Polypropylene gas is then purged into reaction system to keep this living polymerization growing on polypropylene segment to obtain the polypropylene-iodinated polystyrene copolymer.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. A method, comprising:
forming a material including polypropylene and a radiopaque component; and
using the material to form a medical device.

2. The method of claim 1, wherein the medical device includes a mesh.

3. The method of claim 1, wherein the medical device includes a knitted mesh.

4. The method of claim 1, wherein the using the material includes extruding the material to form a filament.

5. The method of claim 1, wherein the forming the material includes forming a plurality of solid pellets of the material.

6. The method of claim 1, wherein the forming the material includes forming a plurality of solid pellets of the material, and the using the material includes placing at least some of the plurality of solid pellets into a mold to form the medical device.

7. The method of claim 1, wherein the medical device includes a molded medical device.

8. The method of claim 1, wherein the radiopaque component includes iodine.

9. The method of claim 1, wherein the forming the material includes compounding polypropylene with the radiopaque component.

10. The method of claim 1, wherein the forming the material includes compounding the polypropylene with barium sulfate.

11. The method of claim 1, wherein the forming the material includes compounding the polypropylene with barium sulfate to form a plurality of solid pellets.

12. The method of claim 1, wherein the forming the material includes forming a polypropylene-iodinated polystyrene co-polymer.

13. The method of claim 1, wherein the material includes a co-polymer having the following formula 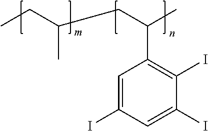
where m and n are integers.

14. The method of claim 1, wherein the forming the material includes copolymerizing propylene with iodinated styrene.

15. The method of claim 1, wherein the forming the material includes copolymerizing propylene with iodinated styrene utilizing a catalyst.

16. A method, comprising:
forming a co-polymer having the following formula 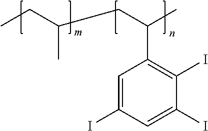
where m and n are integers.

17. The method of claim 16, wherein the forming the co-polymer includes copolymerizing propylene with iodinated styrene.

18. The method of claim 16, wherein the forming the co-polymer includes copolymerizing propylene with iodinated styrene utilizing a catalyst.

19. The method of claim 16, further comprising:
using the co-polymer to form a medical device.

20. A material, comprising:
a co-polymer having the following formula 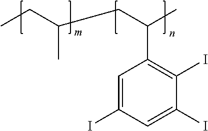
where m and n are integers.
